(19) United States
(12) Patent Application Publication
(10) Pub. No.: US 2021/0345603 A1
(43) Pub. Date: Nov. 11, 2021

(54) COMPOSITION INTENDED FOR INCREASING THE VELOCITY OF SPERMS COMPRISING SEROTONIN AS ACTIVE COMPOUND

(71) Applicants: YILDIZ TEKNÍK ÜNIÌVERSÍTESÌ, Istanbul (TR); BIOSERATECH SAGLIK URUNLERI LTD. STI., Istanbul (TR)

(72) Inventor: Yasemin YILMAZER, Istanbul (TR)

(21) Appl. No.: 17/278,358

(22) PCT Filed: Sep. 26, 2019
(86) PCT No.: PCT/TR2019/050796

§ 371 Date: Mar. 22, 2021

(30) Foreign Application Priority Data
Sep. 27, 2018 (TR) 2018/14102

(51) Int. Cl. A01N 1/02; C12N 5/076

(52) U.S. Cl. CPC A01N 1/0226; C12N 5/061

(57) ABSTRACT

The present invention relates to a composition comprising serotonin which is to be added into the wash solution for incubating the sperms during and after washing, intended for increasing the sperm velocity in insemination and in vitro fertilization methods to select healthy sperms and increase the success in fertilization, said invention discloses the composition increasing the sperm velocity, comprising 8 parts of serotonin, 10 000 parts of vitamin B9, 10 000 parts of vitamin B12, 10 000 parts of vitamin C, 10 000 parts of vitamin D, 10 000 parts of vitamin E, 10 000 parts of zinc mineral and 10 000 parts of selenium mineral by weight in an HTF based sperm wash medium containing Hepes buffer, and the preparation steps thereof.

### TECHNICAL FIELD

The present invention relates to preparing a sperm wash solution to be used in sperm preparation, comprising specific compounds such as serotonin, vitamin D and selenium, on the purpose of selection of the healthy sperm cells with highest motility to be used in the assisted reproductive techniques (ART) and thereby increasing the success of insemination and therapy.

### STATE OF THE ART

Infertility is defined as the failure to conceive after one year of regular unprotected intercourse. Approximately one out of five couples throughout the world experience difficulty to conceive in some period in their lives. According to the data of 2010 reported by the World Health Organization 15% of the couples, namely 48.5 millions of couples face with infertility. As reported by the Turkish Ministry of Health, 2 millions of couples in Turkey have infertility problems. Studies show that in nearly 50% of the cases the cause relates to male infertility and that nearly 25% of said cases relate directly to the sperm quantity and quality.

Preferred methods of therapy in male factor infertility are intrauterine insemination or in vitro fertilization according to the severity of the problem. According to a report published in the European Society of Human Reproduction and Embryology meeting held in 2015, in average 1.5 millions of in vitro fertilization treatment is carried out throughout the world and as a result nearly 350 thousands of healthy babies may be born. In certain couples, inadequacies in the number and in the motility of sperms reduce the chance of pregnancy significantly and treatment must be repeated to achieve success.

In order to process the semen specimen and to select the healthy sperms in insemination and in vitro fertilization methods, the most preferred methods are Swim up and Gradient washing respectively. In cases where the semen specimen contains sperm cells in little amounts, of reduced motility and/or viability, in order to increase the sperm motility and thereby the fertilization, additional agents may be used in the process, such as pentoxifylline (PF) and platelet activating factor (PAF). It has been reported that by means of said agents the sperms achieved a hyperactive like motility and that the rate of the acrosome (an organelle developed over the head part of the spermatozoa) reaction and fertilization increased. Besides the studies reporting these positive effects of the PF and PAF, there are also some studies showing the damaging effects of the same on the sperm membrane and the sperm DNA.

One of the alternative agents currently used to increase motility and fertilization in insemination and in in vitro fertilization is pentoxifylline (PF) and phosphodiesterase inhibitor. It increases the endogenous adenosine triphosphate (ATP) production by increasing the intracellular cyclic adenosine monophosphate (cAMP) levels and glycolysis. It has been reported that the motile spermatozoa in the semen reached to hyperactive like motility in appropriate PF concentrations and that the velocity increased the flagellum motility and the PF treatment increased the fertilization rates in cases where there is acrosome reaction insufficiency. In contrast to the studies showing these positive effects of pentoxifylline, some studies report that the PF has negative effects on the embryo development. Damages in the membranes and nuclei of the sperms, mitochondrial loss and granules in the chromatins have been observed in sperms treated with PF.

Platelet activating factor (PAF) which is another agent used with respect to motility has an effect particularly on the motile spermatozoa. A potent signal found in the spermatozoon is phospholipids. It increases the quality of motility, capacitation, acrosome reaction and ability of fertilization. Studies demonstrate that besides these positive effects of PAF there are damages in the sperm DNA and its effect on the spermatozoon motility is still controversial.

### DESCRIPTION OF THE INVENTION

The aim of the invention is to prepare a sperm wash solution to be used in sperm preparation, comprising specific compounds such as serotonin, vitamin D and selenium, on the purpose of selection of the healthy sperm cells with highest motility to be used in the assisted reproductive techniques (ART) and thereby increasing the success of insemination and therapy.

Since serotonin is naturally produced in the human body, when it's added into the sperm wash solution a natural product will be obtained instead of a product containing synthetic molecules such as PF and PAF. Novel solution to be obtained will be a domestic and an affordable product. The most important thing is that there is no evidence that serotonin causes harm on the animal and human sperm cells. Number of scientific publications demonstrates that serotonin has positive effects on the motility of animals and sperms and on reproductive health. For instance it has been demonstrated that serotonin increased the sperm motility and the insemination rate in the invertebrate animals (Parisi, E., De Prisco P., Capasso A., del Prete M. Serotonin and sperm motility. Cell Biology International Reports. 1984; 8:95.10.1016/0309-1651(84)90075-4 [PubMed: 6546900]). A study performed on mice confirms that serotonin is effective over the head of the sperm (Fujinoki M. Serotonin-enhanced hyperactivation of hamster sperm. Reproduction. 2011; 142:255-266.10.1530/REP-11-0074 [PubMed: 21555358].). In humans, it is effective on the contraction of vas deferens, the regulation of the testicular blood flow, the development of the testicles and the testosterone production (Collin O., Damber J E., Bergh A. 5-Hydroxytryptamine a local regulator of testicular blood flow and vasomotion in rats. Journal of Reproduction and Fertility. 1996; 106:17-22.10.1530/jrf.0.1060017 [PubMed: 8667341].—Trejo F J., Rodriguez M T, Queiroz D B, Padilla P, Avellar M C, Manzano P R, Manjarrez-Gutiérrez Gutiérrez GO. Serotonin concentration, synthesis, cell origin, and targets in the rat caput epididymis during sexual maturation and variations associated with adult mating status: morphological and biochemical studies. J Androl.2007 January-February; 28(1):136-49.). It has been demonstrated that in adolescent males the serotonin levels of the epididymis region increase (Trejo, F J., Rodriguez, M T., Cerbón, M., Kuhn D M, Gutiérrez G M, Mendoza-Rodriguez C., and Picazo, O. Evidence of 5-HT components in human sperm: implications for protein tyrosine phosphorylation and the physiology of motility. Reproduction. 2012 December; 144(6): 677-685. doi:10.1530/REP-12-0145).

It has been confirmed that the fruit flies which are incapable of mating show 25% of mating rate after being fed with serotonin. (Nature Communications, 2016 Dec. 13; 7:13792. DOI: 10.1038/ncomms 13792).

Composition of the present invention which contains serotonin, vitamins B9, B12, C, D, E, minerals such as zinc and selenium in a sperm wash medium (HTF based Hepes buffer) is intended for increasing the velocity and number of sperms in a laboratory medium. In the insemination method, after obtaining the semen specimen, the sperm cells are collected, the healthy and motile ones are selected and inserted into the uterus. As to the in vitro fertilization method, healthy eggs from the female and healthy sperms from the male are fertilized in laboratory conditions and the embryo is transferred to the uterus. In both of the methods, after being washed, the sperms are going to be incubated in a washing solution containing serotonin in a determined dosage and other compounds for a certain period. As serotonin and the other vitamins and minerals are natural, this will not give harm to the cell.

### DETAILED DESCRIPTION OF THE INVENTION

The present invention provides a solution to be used in the insemination and in vitro fertilization methods, in which solution the sperms are incubated during and/or after the washing process in an attempt to increase the success rate of fertilization by selecting the healthy sperms, the solution being intended for increasing the velocity of the sperms and comprising compounds which increase the motility of the sperms; the washing solution comprising 10 000 parts of vitamin D, 10 000 of selenium, 9 parts of serotonin. By using the product for the sperm preparation in insemination and in vitro fertilization methods, an increase in the healthy sperm counts and sperm motilities has been achieved in the specimens obtained after the procedures.

The solution according to present invention in which the sperms are incubated during and/or after the washing process and comprising compounds which increase the motility of the sperms comprises 10 000 parts by weight zinc mineral in the washing solution. Zinc mineral is provided as an excipient in the composition of the invention.

The solution according to present invention in which the sperms are incubated during and/or after the washing process and comprising compounds which increase the motility of the sperms comprises 10 000 parts by weight vitamin C and 10 000 parts by weight vitamin E in the wash solution.

Another characteristic of the solution according to present invention in which the sperms are incubated during and/or after the washing process and comprising compounds which increase the motility of the sperms is that it comprises 10 000 parts by weight vitamin B9 and 10 000 parts by weight vitamin B12 in the washing solution.

The solution which is defined as the sperm wash medium (HTF based containing Hepes buffer) is the solution containing the composition in which there is serotonin, vitamin B9, vitamin B12, vitamin C, vitamin D, vitamin E, zinc mineral and selenium mineral.

The method of manufacturing the composition of the present invention, said composition being intended for increasing the velocity of the sperms to be added into the basic sperm wash solution in which the sperms are incubated, comprises the steps of:
preparing a solution with the relevant amounts of serotonin powder in a basic sperm wash solution,
adding excipients,
mixing the composition.

Serotonin is the active compound of the composition/solution according to present invention. Another characteristic of the present invention is that it comprises serotonin within a range of 25 μM-250 μM. An embodiment of the invention uses 25-250 μM of serotonin. 25 μM, 50 μM, 100 μM and 250 μM serotonin and four distinct compounds as excipients have been used in the studies.

Vitamin B9, vitamin B12, vitamin C, vitamin D, vitamin E, zinc mineral and selenium mineral have been used as excipients in the product of the invention.

In preparing and storing the composition which comprises serotonin increasing the velocity of sperms and the sample composition used in the study, following steps have been carried out:
The active compound serotonin, which is obtained from an African shrub species Griffonia simplicifolia, is available as provided by certain corporations. A solution is prepared with the relative amounts of serotonin obtained from the corporation. 25-250 μM is used in the present invention. In the studies, a solution is prepared with an HTF based sperm wash medium comprising 10 ml of Hepes buffer in 25 μM, 50 μM, 100 μM and 200 μM forms.
Afterwards, according to said ranges relative amounts of vitamin D and selenium are added into the solution.
Zinc mineral, vitamin C, vitamin E, vitamin B9, vitamin B12 can further be added into the composition of the present invention.
Obtained solution is kept in the refrigerator at 4° C. and taken out to room temperature (25° C.) 1 hour prior to use.
Temperature of the solution has an effect on the sperm velocity.
In each use, the solution taken out from the refrigerator is vortexed at 4000 rpm. 2 ml of solution is taken and transferred into sealed containers of 1 cm in diameter.
The semen specimen obtained from the patient is left to liquefy at 37′C.
The semen is washed with HTF based sperm wash medium containing Hepes buffer.
The upper layer is discarded and the sperms at the bottom are incubated with the wash solution comprising serotonin.
If the sperm count is greater than 50 millions then 500 μl of washed sperms are taken, is the sperm count is lower than 50 millions then 1000 μl of sperms are pipetted and taken into previously divided serotonin solution by charging and discharging a couple of times.
After being kept at the room temperature, the sperms the velocities of which are increased are used for insemination and in vitro fertilization procedures.

In the composition of the present invention intended for increasing the sperm velocity, 25 μM-250 μM of serotonin is used preferably.

Following procedures have been used in collecting the sperm cells, developing the cell culture, in the cell count and sperm motility tests, and in examining the interactions of serotonin and the excipients.

The first step is to collect the sperm cells and the semen of the infertile men within the range of 28-45 is used. Specimens are obtained after a sexual abstinence for 3-4 days. All semen specimens are collected in sterile containers and left to liquefy at 37° C. for 30 minutes. 10 μl of sample is taken into Makler® counting chamber and the concentrations, motilities, viabilities, morphologies of the sperms and the leukocyte existence are evaluated. According to the motility results, the semen specimen is prepared by being washed with HTF based sperm wash solution containing Hepes buffer by using concentration and gradient methods. Washed and prepared sperms are collected in the sperm wash medium in which certain amounts of (25, 50, 100 and 200 μM) 5-HT was added, and incubated at 25′C in certain periods of time (30, 60, 90 and 120 minutes).

In the cell count step, the sperm cell count is repeated twice for the specimens Incubated according to certain time periods. After being thoroughly mixed, 10 μl of sperm specimen is evaluated in a Makler® counting chamber.

Sperm motility tests are performed with the sperms kept in a serotonin medium, by the measurements in the Makler® counting chamber with 30, 60, 90 and 120 min. intervals. Samples with a sperm concentration of 2×10⁶ and 50×10⁵ per ml max. are included in the analyses. As to the interactions of serotonin and the excipients, washed sperm cells are taken into the sperm wash mediums containing 5-HT and vitamin D, serotonin, selenium, zinc mineral, vitamin C, vitamin E, vitamin B9, vitamin B12 in certain amounts respectively, and are incubated for certain time intervals in an incubator. The dosage of the compounds is determined by examining the effect of the same on sperm motility, both individually and in combination.

As a result of these studies, it has been observed that a significant increase in the sperm motilities is achieved with the composition comprising the mentioned ratios of the compounds.

In the sperms obtained from the male patients with a sperm count lower than 60 millions and with a sperm velocity of 15-30%, an Increase of 20% in both sperm number and velocity has been achieved by incubation with the product of the present invention. The developed product is intended for intrauterine insemination and in vitro fertilization in laboratory conditions.

Table 1 shows the results of the sperm cells incubated at 25° C. for certain time intervals (30, 60, 90 and 120 minutes) after being collected in a sperm wash medium in 25, 50, 100 and 200 μM serotonin and 5-HT was added. Obtained results for the change in motility (sperm velocity) has been listed in Table 1.

Change in Average Motility

| Period | 30 min | 60 min | 90 min | 120 min |
|---|---|---|---|---|
| After washing | 75 | 75 | 75 | 75 |
| Control | 86.43 | 78.7 | 78.58 | 72.58 |

| | | | | | |
|---|---|---|---|---|---|
| 25 | μM | 82.76 | 86.28 | 81.48 | 81.41 |
| 50 | μM | 80.56 | 81.66 | 86.08 | 80.45 |
| 100 | μM | 83.53 | 80.16 | 83.86 | 80.4 |
| 200 | μM | 82.06 | 86.46 | 86.55 | 82.55 |

1. A solution for increasing the success rate of fertilization by selecting the healthy sperms for the insemination and in vitro fertilization, for increasing the velocity of the sperms and comprising compounds which increase the motility of the sperms; wherein, the wash solution comprises 10,000 parts of vitamin D, 10.000 parts of selenium, 8 parts of serotonin.

2. Solution according to claim 1, for increasing the velocity of the sperms, wherein, it comprises 10,000 parts by weight zinc mineral in the wash solution.

3. Solution according to claim 1 for increasing the velocity of the sperms, wherein, it comprises 10,000 parts by weight vitamin C and 10,000 parts by weight vitamin E in the wash solution.

4. Solution according to claim 3 for increasing the velocity of the sperms, wherein, it comprises 10,000 parts by weight vitamin B9 and 10,000 parts by weight vitamin B12 in the wash solution.

5. Solution according to claim 1 for increasing the velocity of the sperms, wherein, it comprises 25 μM-250 μM serotonin.

6. A method for preparing a solution according to claim 1 for increasing the velocity of the sperms, wherein, it comprises the steps of:
preparing a solution with a relative amount of serotonin,
adding excipients,
mixing the composition.
